(19) United States
(12) Patent Application Publication
(10) Pub. No.: US 2012/0165653 A1
(43) Pub. Date: Jun. 28, 2012

(54) MR imaging system with cardiac coil and defibrillator

(75) Inventors: Steffen Weiss, Hamburg (DE); Bernd David, Huettblek (DE); Oliver Lips, Hamburg (DE); Sascha Krueger, Hamburg (DE)

(73) Assignee: KONINKLIJKE PHILIPS ELECTRONICS N.V., Eindhoven (NL)

(21) Appl. No.: 13/391,849

(22) PCT Filed: Sep. 15, 2010
(86) PCT No.: PCT/IB10/54155

§ 371 Date: Feb. 23, 2012

(30) Foreign Application Priority Data
Sep. 21, 2009 (EP) 09170832.1

(51) Int. Cl. A61B 5/055; A61N 1/39; A61N 1/04

(52) U.S. Cl. 600/411

(57) ABSTRACT

The invention relates to a magnetic resonance imaging system comprising a main magnet coil (2) for generating a uniform, steady magnetic field within an examination volume, a number of gradient coils (4, 5, 6) for generating switched magnetic field gradients in different spatial directions within the examination volume, at least one cardiac RF coil (11) for transmitting RF pulses to and/or receiving MR signals from the chest region of a body (10) of a patient positioned in the examination volume, a control unit (13) for controlling the temporal succession of RF pulses and switched magnetic field gradients, and a reconstruction unit (15) for reconstructing a MR image from the MR signals. In order to enable quick and safe defibrillation at any time during a MR imaging procedure, the invention proposes that at least one opening (19, 22) is provided in the cardiac RF coil (11), through which opening (19, 22) a portion of the skin surface in the chest region of the body (10) is accessible, wherein the magnetic resonance imaging system further comprises a defibrillator unit (17) connected to at least one defibrillator electrode (23) fitting through the at least one opening (19, 22) provided in the cardiac RF coil (11). Alternatively, the invention proposes that at least one defibrillator cable (30) is affixed to the cardiac RF coil (11), wherein the defibrillator unit (17) is connectable to at least one defibrillator electrode pad (26) via the at least one defibrillator cable (30).

### FIELD OF THE INVENTION

The invention relates to the field of magnetic resonance (MR) imaging. It concerns a MR imaging system comprising a cardiac RF coil and a defibrillator unit. The invention also relates to a cardiac RF coil adapted to be used with a defibrillator unit.

Image-forming MR methods which utilize the interaction between magnetic fields and nuclear spins in order to form two-dimensional or three-dimensional images are widely used nowadays, notably in the field of medical diagnostics, because for the imaging of soft tissue they are superior to other imaging methods in many respects, do not require ionizing radiation and are usually not invasive.

### BACKGROUND OF THE INVENTION

According to the MR method in general, the body of the patient to be examined is arranged in a strong, uniform magnetic field whose direction at the same time defines an axis (normally the z-axis) of the co-ordinate system on which the measurement is based. The magnetic field produces different energy levels for the individual nuclear spins in dependence on the magnetic field strength which can be excited (spin resonance) by application of an electromagnetic alternating field (RF field) of defined frequency (so-called Larmor frequency, or MR frequency). From a macroscopic point of view the distribution of the individual nuclear spins produces an overall magnetization which can be deflected out of the state of equilibrium by application of an electromagnetic pulse of appropriate frequency (RF pulse) while the magnetic field extends perpendicular to the z-axis, so that the magnetization performs a precessional motion about the z-axis.

The variation of the magnetization can be detected by means of receiving RF coils which are arranged and oriented within an examination volume of the MR device in such a manner that the variation of the magnetization is measured in the direction perpendicular to the z-axis.

In order to realize spatial resolution in the body, linear magnetic field gradients extending along the three main axes are superposed on the uniform magnetic field, leading to a linear spatial dependency of the spin resonance frequency. The signal picked up in the receiving coils then contains components of different frequencies which can be associated with different locations in the body. The signal data obtained via the receiving coils corresponds to the spatial frequency domain and is called k-space data. The k-space data usually includes multiple lines acquired with different phase encoding. Each line is digitized by collecting a number of samples. A set of k-space data is converted to an MR image, e.g., by means of Fourier transformation.

Cardiac interventional MR imaging is a promising tool in which accurate localization of an interventional instrument with excellent soft tissue contrast can be combined. Moreover, functional information from the heart can be obtained by means of appropriate MR imaging techniques. The combination of MR imaging with tracking of interventional instruments is especially advantageous for therapeutic applications that require therapy monitoring, like, e.g., MR electrophysiology interventions. For all kinds of MR-monitored cardiac interventions, particularly high quality cardiac MR imaging is essential. To this end, multi-element cardiac RF coils are used in state-of-the-art magnetic resonance imaging systems for signal reception in cardiac applications. Such cardiac RF coils consist of 16 to 32 coil elements arranged on a (flexible) coil body. Sometimes, the coil elements are clustered in a posterior and an anterior part. Cardiac interventions, such as, for example, electrophysiology interventions, bear a significant risk of inducing atrial and ventricular tachycardia including fibrillation. Therefore, the patient must be quickly accessible at all times during MR-guided interventions to perform external cardioversion or defibrillation. For this reason, a defibrillator unit is used in combination with the magnetic resonance imaging system. The defibrillator unit directs a pulse of electrical direct current into the patient's heart to return it to its regular rhythm. To deliver such a pulse of electrical current to the heart, either adhesive defibrillator electrode pads or defibrillator electrodes arranged on handheld paddles that are connected with the defibrillator unit are used. Self-adhesive defibrillator electrode pads are fixedly attached on the chest area of the patient. Handheld defibrillator paddles are usually applied manually in anterior-apex configuration on the chest area of the patient in an emergency situation for correcting a condition of fibrillation.

A major problem of presently existing systems is that the defibrillator paddle positions are incompatible with the position of standard cardiac RF coils. In case of emergency, the patient has to be removed from the examination volume of the MR imaging system and the cardiac RF coil must be detached from the chest area of the patient before the defibrillator paddles can be applied. This procedure requires a significant amount of time. However, a quick defibrillation is required in a condition of fibrillation in order to avoid serious consequences for the patient's health.

Adhesive defibrillator electrode pads may be attached precautionary to the patient's chest in order to expedite defibrillation therapy to the patient in the event the patient experiences fibrillation during the MR-guided medical procedure. However, adhesive defibrillation pads may interfere with the MR imaging procedure such that it may not be practically feasible to continually couple the patient to the defibrillator unit. Undesirable electromagnetic interactions of the switched magnetic field gradients and RF pulses being part of the imaging procedure with various components of the defibrillator electrode pads may occur. The metal foils forming the electrodes of the defibrillator electrode pads cause RF shielding, and eddy currents may be induced in the metal foils by the switched magnetic field gradients. This results in significant MR image artifacts. Moreover, the irradiated RF pulses may induce currents in the wire leads, via which the defibrillator electrode pads are connected to the defibrillator unit. Dangerous heating of the wire leads can injure the patient.

### SUMMARY OF THE INVENTION

From the foregoing it is readily appreciated that there is a need for an improved MR imaging system. It is consequently an object of the invention to provide a MR imaging system enabling high quality cardiac MR imaging, wherein safe external cardioversion or defibrillation is possible quickly at any time during the MR imaging procedure.

In accordance with the present invention, a MR imaging system for cardiac applications is disclosed. The system comprises:

a main magnet coil for generating a uniform, steady magnetic field within an examination volume,

a number of gradient coils for generating switched magnetic field gradients in different spatial directions within the examination volume,

at least one cardiac RF coil for transmitting RF pulses to and/or receiving MR signals from the chest region of a body of a patient positioned in the examination volume, wherein at least one opening is provided in the cardiac RF coil, through which opening a portion of the skin surface in the chest region of the body is accessible,

a defibrillator unit connected to at least one defibrillator electrode fitting through the at least one opening provided in the cardiac RF coil,

a control unit for controlling the temporal succession of RF pulses and switched magnetic field gradients, and

a reconstruction unit for reconstructing a MR image from the MR signals.

The magnetic resonance imaging system according to the invention comprises a defibrillator unit connected to (usually two) defibrillator electrodes fitting through openings in the cardiac RF coil placed on the chest of the examined patient. The cardiac RF coil of the magnetic resonance imaging system according to the invention provides access to the patient's skin in the chest region at the required defibrillation locations. This enables a safe defibrillation at any time during a MR-guided cardiac intervention. In particular, because of the openings in the cardiac RF coil there is no necessity to detach the cardiac RF coil from the chest of the patient for defibrillation in a case of emergency.

Moreover, the invention proposes to use defibrillator electrodes that are shaped corresponding to the shape of the openings in the cardiac RF coil. In this way, it is made sure that the defibrillator electrodes, which may for example be arranged on handheld paddles, fit exactly into the openings of the cardiac RF coil.

Preferably, the cardiac RF coil of the magnetic resonance imaging system according to the invention is an array coil comprising two or more coil elements each having the form of conductor loops. As mentioned above, conventional cardiac RF coils comprise 16 to 32 conductor loops as coil elements. Two or more openings may be provided in the cardiac RF coil within regions enclosed by the conductor loops of adjacent coil elements. The body and/or the packaging of the cardiac RF coil have of course to be provided with corresponding openings as well such that the defibrillation locations on the chest of the patient are accessible. Two or more defibrillator electrodes may be arranged on a paddle of the defibrillator unit in such a manner that the defibrillator electrodes fit trough the two or more openings provided in the cardiac RF coil, i.e. through the respective open conductor loops of the coil elements. The defibrillator electrodes may be attached to the paddles via elastic elements establishing a safe electrical contact by pressing the defibrillator electrodes reaching through the openings in the cardiac RF coil against the skin surface of the body of the patient. The components of the defibrillator paddles should of course be constructed from non-ferromagnetic materials to be safely operable in the MR imaging environment.

In accordance with a further aspect of the invention, adhesive defibrillator electrode pads connectable to the defibrillator unit via defibrillator cables may be used. In this variant of the invention, the defibrillator cables are affixed to the cardiac RF coil of the magnetic resonance imaging system. The defibrillator electrode pads have to be connected to the defibrillator unit via low impedance cables which are prone to RF-induced heating. Such heating effects can be suppressed by providing per se known resonant RF cable traps on the defibrillator cables. However, the cable traps become hot themselves during RF irradiation. By affixing the defibrillator cables to the cardiac RF coil, a cable routing is provided that avoids a close contact between the skin of the patient and the defibrillator cable and the cable traps. Hence, this variant of the invention also enables quick and safe defibrillation at any time during a MR-guided intervention without the risk of injury of the patient. In this context, it has to be considered that all cables present in the cardiac RF coil, including the defibrillator cables as well as the RF cables connecting the coil elements of the cardiac RF coil, exhibit mutual RF coupling. The coupling depends strongly on the routing geometry of the cables. The invention allows a fixed geometry of the complete cabling of the cardiac RF coil and of the positions of the cable traps. This geometry can be optimized once for efficiency and safety.

According to a preferred embodiment of the invention, the defibrillator cables comprise externally accessible connectors for releasably connecting the defibrillator cables with the defibrillator electrode pads. In this embodiment, the connectors define fixed connection sites between the integrated defibrillator cables and the defibrillator electrode pads. Small feed-through gaps may be provided in the body of the cardiac RF coil. Each adhesive defibrillator electrode pad may be equipped with one or more short cable stubs terminated by a connector compatible with the connectors provided on the integrated defibrillator cables of the cardiac RF coil.

According to another preferred embodiment of the invention, the adhesive defibrillator electrode pads are constructed in such a manner that RF-induced or gradient-induced circular currents and resulting MR image artefacts are avoided. Each defibrillator electrode pad comprises one or more electrode foils that are formed in a pattern that avoids closed current paths. In this way, undesirable induced circular currents can be suppressed without inhibiting the current flow as required for defibrillation. The pattern of the electrode foil can be selected such that a relatively homogeneous distribution of the defibrillation current over the area of the pad is provided. Skin irritations by the defibrillation currents are prevented in this way. To this end, the pattern of the electrode foil may include a plurality of elongate sections extending radially outward from a centre. Such a generally star-shaped pattern is well suited for a defibrillation electrode pad according to the invention.

According to yet another preferred embodiment of the invention, the defibrillator unit is connectable to at least two defibrillator electrode pads via at least two defibrillator cables, wherein the defibrillator unit is configured to measure the impedance between the at least two defibrillator electrode pads. This configuration of the defibrillator unit enables the measurement of the impedance between the adhesive pads at regular intervals during the entire interventional MR imaging procedure. If the impedance is outside of a predefined range, the defibrillator unit may issue an alarm. Loosening of one of the electrode pads or the corresponding electrical connections can effectively be detected by measuring the impedance.

During an MR-guided cardiac intervention, the patient should be quickly removable from the examination volume of the MR imaging system and free access to the patient should be possible within short time. In an emergency situation, the intervention needs to be stopped immediately, for example in order to commence surgery or cardiopulmonary resuscitation. For this reason, the cardiac RF coil must be quickly removable from the patient at all times. Therefore, the cardiac RF coil should be constructed such that at least an anterior part of the cardiac RF coil is fastened to the posterior part and/or to the patient by a mechanism that can simply and quickly be released. Also the electrical connections connecting the adhesive defibrillator electrode pads to the integrated defibrillator cables of the cardiac RF coil should be constructed to release quickly at low force. For example, snap-fastener connections are well-suited for this purpose.

The enclosed drawings disclose preferred embodiments of the present invention. It should be understood, however, that the drawings are designed for the purpose of illustration only and not as a definition of the limits of the invention.

### BRIEF DESCRIPTION OF THE DRAWINGS

In the drawings

FIG. 1 schematically shows a MR imaging system according to the invention;

FIG. 2 is a sketch of a cardiac RF coil according to the invention;

FIG. 3 illustrates a defibrillator paddle to be used in connection with the cardiac RF coil shown in FIG. 2;

FIG. 4 is a cut side view of a cardiac RF coil placed on the chest of a patient's body in combination with an adhesive defibrillator pad;

FIG. 5 is a top view of the cardiac RF coil shown in FIG. 4.

FIG. 6 illustrates defibrillator electrode patterns according to the invention.

### DETAILED DESCRIPTION OF EMBODIMENTS

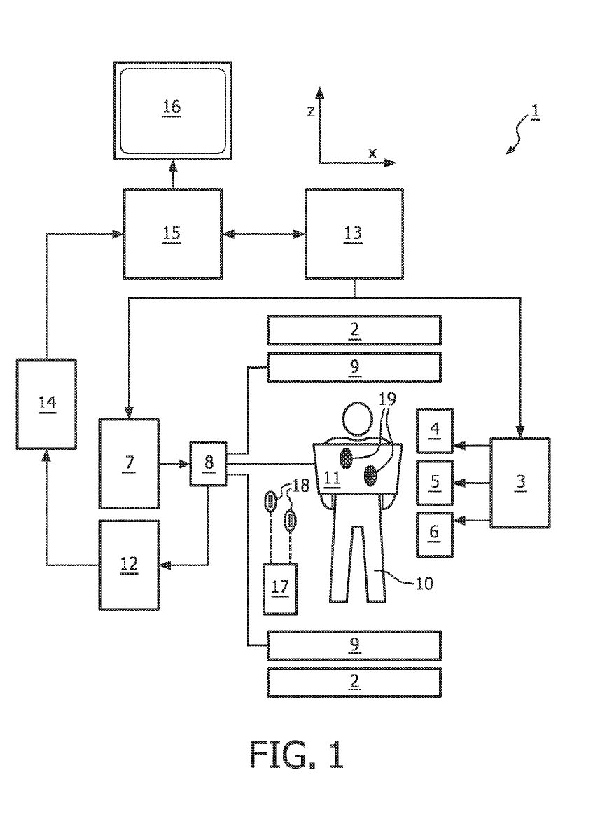

With reference to FIG. 1, a MR device 1 is shown. The device comprises superconducting or resistive main magnet coils 2 such that a substantially uniform, temporally constant main magnetic field is created along a z-axis through an examination volume.

A magnetic resonance generation and manipulation system applies a series of RF pulses and switched magnetic field gradients to invert or excite nuclear magnetic spins, induce magnetic resonance, refocus magnetic resonance, manipulate magnetic resonance, spatially and otherwise encode the magnetic resonance, saturate spins, and the like to perform MR imaging.

More specifically, a gradient pulse amplifier 3 applies current pulses to selected ones of whole-body gradient coils 4, 5 and 6 along x, y and z-axes of the examination volume. A RF transmitter 7 transmits RF pulses or pulse packets, via a send-/receive switch 8, to a whole-body volume RF coil 9 to transmit RF pulses into the examination volume. A typical MR imaging sequence is composed of a packet of RF pulse segments of short duration which taken together with each other and any applied magnetic field gradients achieve a selected manipulation of nuclear magnetic resonance. The RF pulses are used to saturate, excite resonance, invert magnetization, refocus resonance, or manipulate resonance and select a portion of a body 10 positioned in the examination volume. The MR signals are also picked up by the whole-body volume RF coil 9.

For generation of MR images of the patient's heart and the coronary vessels, a cardiac RF coil 11 is placed contiguous to the region selected for imaging. In practical embodiments, the cardiac RF coil 11 may comprise a posterior part and an anterior part. Only the anterior part of the cardiac RF coil 11 placed directly on the chest of the body 10 of the patient is depicted in FIG. 1. The cardiac RF coil 11 can be used to receive MR signals induced by body-coil RF transmissions.

The resultant MR signals picked up by the whole body volume RF coil 9 and/or the cardiac RF coil 11 are demodulated by a receiver 12 preferably including one or more pre-amplifiers (not shown). The receiver 12 is connected to the RF coils 9, 11 via send-/receive switch 8.

A host computer 13 controls the gradient pulse amplifier 3 and the transmitter 7 to generate any of a plurality of MR imaging sequences, such as turbo spin echo (TSE) imaging, echo planar imaging (EPI), and the like. For the selected sequence, the receiver 12 receives a single or a plurality of MR data lines in rapid succession following each RF excitation pulse. A data acquisition system 14 performs analog-to-digital conversion of the received signals and converts each MR data line to a digital format suitable for further processing. In modern MR imaging systems, the data acquisition system 14 is a separate computer which is specialized in acquisition of raw image data.

Ultimately, the digital raw image data is reconstructed into an image representation by a reconstruction processor 15 which applies a Fourier transform or other appropriate reconstruction algorithms. The MR image may represent a planar slice through the patient, an array of parallel planar slices, a three-dimensional volume, or the like. The images then stored in an image memory where it may be accessed for converting slices, projections, or other portions of the image representation into appropriate format for visualization, for example via a video monitor 16 which provides a man-readable display of the resultant MR image.

Provision is made for a defibrillator unit 17 connected to two handheld defibrillator paddles 18. The defibrillator paddles 18 can be applied any time during a MR imaging scan in anterior-apex configuration to the chest region of the body 10 of the patient in order to correct a condition of fibrillation. To this end, the defibrillator unit 17 generates a current pulse which is directed into the heart of the patient. In principle, a defibrillator device of conventional type can be used as a defibrillator unit of the MR imaging system of the invention.

The cardiac RF coil 11 has two openings 19, through which the defibrillation locations at the skin surface of the body 10 are accessible. The shape of the openings 19 matches the shape of the defibrillator paddles 18 such that the defibrillator electrodes attached to the defibrillator paddles 18 reach through the openings 19 and establish electrical contact with the patient's skin.

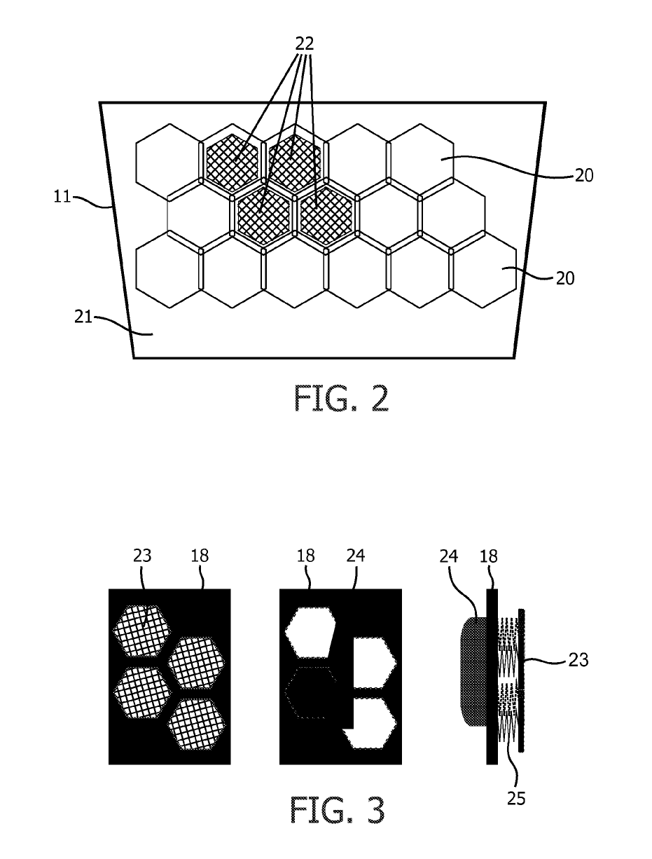

With reference to FIG. 2, an embodiment of the cardiac RF coil 11 according to the invention is described in more detail. The cardiac RF coil 11 is an array coil comprising sixteen coil elements 20 each having the form of conductor loops. The coil elements 20 are arranged on a flexible coil body 21. Four openings 22 are provided in the cardiac RF coil 11 in the form of gaps in the coil body 21 within regions enclosed by the conductor loops of the respective four adjacent coil elements 20. For reasons of simplicity, the further elements of the cardiac RF coil, such as RF electronics and cabling, are not depicted in FIG. 2.

FIG. 3 shows (from left to right) a bottom view, a top view and a side view of the defibrillation paddle 18. Four defibrillator electrodes 23 are arranged on the paddle 18, wherein the shape and the arrangement of the defibrillator electrodes 23 is selected such that the defibrillator electrodes 23 fit through the four openings 22 provided in the cardiac RF coil 11 as shown in FIG. 2. The defibrillator paddle 18 comprises a handle 24 for manually placing the defibrillator paddle 18 in the correct position over the cardiac RF coil 11 such that the electrodes 23 reach through the openings 22. The defibrillator electrodes 23 are attached to the paddle 18 via elastic springs 25 pressing the defibrillator electrodes 23 reaching through the openings 22 against the skin surface of the body 10 of the patient. Again for reasons of simplicity, the cabling connecting the defibrillator electrodes 23 to the defibrillator unit 17 is not depicted in FIG. 3.

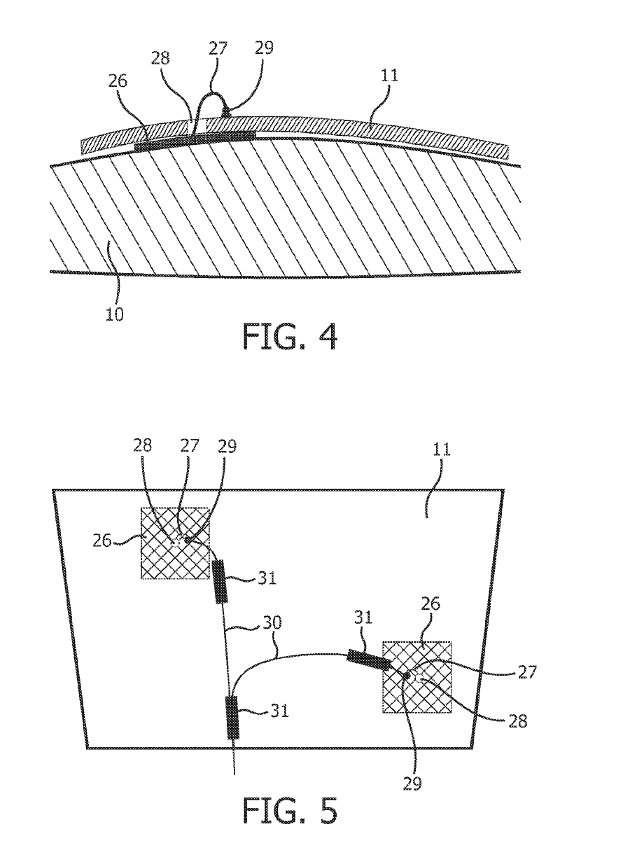

With reference to FIG. 4, an alternative solution is described. FIG. 4 schematically shows a cut side view of the cardiac RF coil 11 placed on the chest of the patient's body 10. An adhesive defibrillator electrode pad 26 is attached to the patient's chest. The cardiac RF coil 11 is placed on top of the defibrillator electrode pad 26. The adhesive pad 26 is equipped with a short cable stub 27 for establishing the required electrical connection. The cable stub 27 is guided through a small opening 28 in the cardiac RF coil 11. The cardiac RF coil 11 comprises an externally accessible electrical connection site 29, which can for example be a conventional snap-fastener connector. The cardiac RF coil 11 incorporates defibrillator cables (not depicted in FIG. 4) for connecting the adhesive defibrillator electrode pad 26 to the defibrillator unit 17. The snap-fastener connector 29 enables releasable connection of the defibrillator cable with the defibrillator electrode pad 26.

When using the arrangement shown in FIG. 4, the defibrillator electrode pad 26 will firstly be fixed on the patient's chest. Thereafter, the cardiac RF coil 11 will be positioned on top of the defibrillator electrode pad 26, wherein the cable stub 27 is fed through the gap 28. Finally, the cable stub 27 is snapped onto the connector 29 in order to establish the electrical connection with the pad 26.

FIG. 5 is a top view of the cardiac RF coil 11 shown in FIG. 4. FIG. 5 shows the defibrillator cables 30 that establish electrical connection with the pads 26 via the snap-fastener connectors 29. The defibrillator cables 30 are firmly affixed to the cardiac RF coil 11 in order to achieve a fixed relative geometry of the cabling within the cardiac RF coil 11. The coil elements of the cardiac RF coil 11 as well as the RF electronics and RF cabling are not shown in FIG. 5. Resonant cable traps 31 are provided on the defibrillator cables 30 in order to avoid RF-induced heating of the cables. The defibrillator cables 30 as well as the cable traps 31 are positioned within the cardiac RF coil 11 in such a manner that a contact with the patient's skin is prevented.

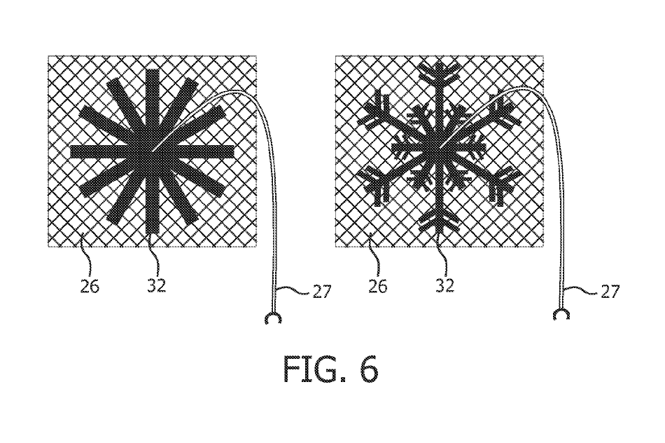

FIG. 6 illustrates different electrode patterns of the adhesive defibrillation electrode pads 26 to be used in the embodiments shown in FIGS. 4 and 5. FIG. 6 shows bottom views of adhesive pads 26 with two different electrode patterns. Electrode foils 32, such as, for example, copper foils, are applied to the bottom of the flexible, electrically non-conducting plastic or paper body of the pads 26. An electrically conductive gel is applied on the bottom side of the adhesive pads 26. FIG. 6 shows that the electrode foils 32 are formed in patterns that avoid closed current paths. In this way, the induction of currents by RF irradiation and/or gradient switching can be avoided. The patterns are generally star-shaped and include a plurality of elongate sections extending radially outward from a centre where the cable stubs 27 are connected to the electrode foils. The application of sufficient defibrillation currents to the skin of the patient is not impeded by the patterns shown in FIG. 6.

1. Magnetic resonance imaging system comprising:
a main magnet coil (2) for generating a uniform, steady magnetic field within an examination volume,
a number of gradient coils (4, 5, 6) for generating switched magnetic field gradients in different spatial directions within the examination volume,
at least one cardiac RF coil (11) for transmitting RF pulses to and/or receiving MR signals from the chest region of a body (10) of a patient positioned in the examination volume, wherein at least one opening (19, 22) is provided in the cardiac RF coil (11), through which opening (19, 22) a portion of the skin surface in the chest region of the body (10) is accessible,
a defibrillator unit (17) connected to at least one defibrillator electrode (23) fitting through the at least one opening (19, 22) provided in the cardiac RF coil (11),
a control unit (13) for controlling the temporal succession of RF pulses and switched magnetic field gradients, and
a reconstruction unit (15) for reconstructing a MR image from the MR signals.

2. Magnetic resonance imaging system according to claim 1, wherein the a least one defibrillator electrode (23) is shaped corresponding to the shape of the at least one opening (19, 22) in the cardiac RF coil (11).

3. Magnetic resonance imaging system according to claim 1, wherein the cardiac RF coil (11) is an array coil comprising two or more coil elements (20) each having the form of conductor loops.

4. Magnetic resonance imaging system according to claim 3, wherein two or more openings (22) are provided in the cardiac RF coil (11) within regions enclosed by the conductor loops of adjacent coil elements (20).

5. Magnetic resonance imaging system according to claim 4, wherein two or more defibrillator electrodes (23) are arranged on a paddle (18) of the defibrillator unit (17) in such a manner that the defibrillator electrodes (23) fit through the two or more openings (22) provided in the cardiac RF coil (11).

6. Magnetic resonance imaging system according to claim 5, wherein the defibrillator electrodes (23) are attached to the paddle (18) via elastic elements (25) pressing the defibrillator electrodes (23) reaching through the openings (22) in the cardiac RF coil (11) against the skin surface of the body (10) of the patient.

7. Magnetic resonance imaging system comprising:
a main magnet coil (2) for generating a uniform, steady magnetic field within an examination volume,
a number of gradient coils (4, 5, 6) for generating switched magnetic field gradients in different spatial directions within the examination volume,
at least one cardiac RF coil (11) for transmitting RF pulses to and/or receiving MR signals from the chest region of a body (10) of a patient positioned in the examination volume, wherein at least one defibrillator cable (30) is affixed to the cardiac RF coil (11),
a defibrillator unit (17) connectable to at least one defibrillator electrode pad (26) via the at least one defibrillator cable (30),
a control unit (15) for controlling the temporal succession of RF pulses and switched magnetic field gradients, and
a reconstruction unit (15) for reconstructing a MR image from the MR signals.

8. Magnetic resonance imaging system according to claim 7, wherein at least one RF cable trap (31) is provided on the defibrillator cable (30), the cable trap (31) being affixed to the cardiac RF coil (11).

9. Magnetic resonance imaging system according to claim 7, wherein the defibrillator cable (30) comprises an externally accessible connector (29) for releasably connecting the defibrillator cable (30) with the defibrillator electrode pad (26).

10. Magnetic resonance imaging system according to claim 7, wherein the defibrillator electrode pad (26) is adhesive.

11. Magnetic resonance imaging system according to claim 7, wherein the defibrillator electrode pad (26) comprises one or more electrode foils (32) formed in a pattern that avoids closed current paths.

12. Magnetic resonance imaging system according to claim 11, wherein the pattern of the electrode foil (32) includes a plurality of elongate sections extending radially outward from a center.

13. Magnetic resonance imaging system according to claim 7, wherein the defibrillator unit (17) is connectable to at least two defibrillator electrode pads (26) via at least two defibrillator cables (30), wherein the defibrillator unit (17) is configured to measure the impedance between the at least two defibrillator electrode pads (26).

14. Cardiac RF coil for transmitting RF pulses to and/or receiving MR signals from the chest region of a body (10) of a patient positioned in the examination volume of a MR imaging system (1), wherein at least one opening (19, 22) is provided in the cardiac RF coil (11), through which opening (19, 22) a portion of the skin surface in the chest region of the body (10) is accessible, the shape of the at least one opening (19, 22) being matched to the shape of a defibrillator paddle (18) in such a manner, that at least one defibrillator electrode (23) of the defibrillator paddle (18) reaches through the at least one opening (19, 22).

15. Cardiac RF coil for transmitting RF pulses to and/or receiving MR signals from the chest region of a body (10) of a patient positioned in the examination volume of a MR imaging system (1), wherein at least one defibrillator cable (30) is affixed to the cardiac RF coil (11) for connecting a defibrillator electrode pad (26) to a defibrillator unit (17), the defibrillator cable (30) comprising an externally accessible connector (29) for releasably connecting the defibrillator cable (30) to the defibrillator electrode pad (26).
